(19) United States
(12) Patent Application Publication
(10) Pub. No.: US 2003/0022274 A1
(43) Pub. Date: Jan. 30, 2003

(54) Partially hydrolysed protein nutrient supplement

(76) Inventor: Marcel C. McNeil, Boucherville (CA)

Correspondence Address: Shapiro Cohen, Station D, PO Box 3440, Ottawa, ON, K1P6P1, CA

(21) Appl. No.: 10/129,551

(22) PCT Filed: Sep. 10, 2001
(86) PCT No.: PCT/CA01/01292

(30) Foreign Application Priority Data
Sep. 11, 2000 (GB) 0022236.4

(51) Int. Cl. C12P 21/06; A23K 1/00; A23L 1/00

(52) U.S. Cl. 435/68.1; 530/370; 426/615

(57) ABSTRACT

A multistep process according for preparing a partially hydrolyzed protein nutrient supplement, including an enzymatic hydrolysis step using a protease enzyme. In the enzymatic hydrolysis step, the degree of hydrolysis is controlled to be about 27%. The degree of hydrolysis is monitored during the enzymatic hydrolysis, and the reaction is terminated by deactivating the enzymes when the desired degree of hydrolysis has been reached. The resulting partially hydrolyzed protein product avoids the objectionable taste and feel properties frequently found with vegetable protein, in particular soy bean protein, hydrolysaes. The partially hydrolyzed protein product is of use in the food industry, and in preparing dietary supplement materials for which purpose the partially hydrolyzed protein can be combined with a range of additives such as natural vitamin E. The partially hydrolyzed protein product provides a range and quantity of protein materials normally only associated with animal proteins, such as red blood cell based products, without using any animal based materials as a source.

This invention is concerned with a process for the preparation of a partially hydrolysed protein nutrient supplement. More particularly, this invention is concerned with a process for the preparation of a hydrolysed protein nutrient supplement for use in food and dietary supplement material manufacture.

Several processes have been proposed for the enzymatic hydrolysis of a number of protein materials of both animal and vegetable origin to provide materials for use mainly in the food industry. The protein material which appears to have received the most attention is soy bean protein.

In CA 604,712, it is proposed to use digested soy bean meal in a fermentation process to obtain l-glutamic acid. In CA 942,992 it is proposed to process soy bean protein materials to form an edible protein product, that has high dispersibility, excellent mouth texture, and excellent mouth feel. This material can be used to make a milk-type product. The process includes an enzymatic hydrolysis step. In CA 997,202 it is proposed to purify soy bean protein, by removing most of the carbohydrates commonly present by the use of carbohydrate specific enzymes. In CA 1,119,485 it is proposed to process a number of materials, including both animal derived and vegetable derived proteins, including albumin, milk whey, soy bean and wheat gluten protein, to provide an organoleptically desirable protein material suitable for use as a dietary supplement. The process includes a hydrolysis step, which is preferably enzymatic. In CA 1,230,515 it is proposed to process vegetable protein, preferably soy bean protein, to provide an enzymatically hydrolysed protein isolate which is suitable for use in cheese products. In CA 1,235,941 it is proposed to process an aqueous solution of soy bean protein to provide a calcium rich bean soup drink. The soy bean protein is hydrolysed enzymatically, and a calcium salt is added to the reaction product. In CA 1,251,988 it is proposed to process vegetable proteins, such as soy bean isolates, for use in the production of a number of foods, including cheese, beverages, and nutrition supplements. The process includes an enzymatic hydrolysis step.

In U.S. Pat. No. 4,757,007, it is proposed to hydrolyse soy bean protein with a hydrolase, and then separate the product into two fractions by the use of aqueous trichloracetic acid. The less soluble fraction is said to be useful as an emulsifier, and the more soluble fraction as a foaming agent. In U.S. Pat. No. 5,427,921 it is proposed to hydrolyse a non-yeast protein with yeast. By appropriate selection of the protein, it is stated that a yeast extract with a specific flavour can be produced for use as a taste additive in the food industry. The nonyeast protein can be soy bean protein. In U.S. Pat. No. 5,618,689 it is proposed to make a vegetable protein hydrolysate product by hydrolysing a substrate, such as soy protein, with a sterile enzyme system. In U.S. Pat. No. 5,663,058 it is proposed to hydrolyse soy bean protein with a hydrolase enzyme. The resulting material is said to be suitable for use in pickling solutions, and in thick materials such as soups. In U.S. Pat. No. 5,716,801 it is proposed to obtain a “well tasting . . . vegetable protein hydrolysate from soy and other materials”, by a process which combines hydrolysis and ultrafiltration. An enzymatic hydrolysis is used.

In practise it has been found that most of the proposed processes for hydrolysing protein material of either vegetable or animal origin, and in particular soy bean protein, suffer from a singular disadvantage. The difficulty is that most of the products do not have an acceptable taste or feel in the mouth when used in foods. This invention seeks to overcome this difficulty, and to provide a partially hydrolysed protein supplement which does not impart an unacceptable taste or flavour to a food or other dietary supplement material.

In the process according to this invention for preparing a partially hydrolysed protein nutrient supplement, several steps are used including an enzymatic hydrolysis step. In the enzymatic hydrolysis step the degree of hydrolysis is controlled to be about 27%. The degree of hydrolysis is monitored during the enzymatic hydrolysis, and the reaction is terminated by deactivating the enzymes when the desired degree of hydrolysis has been reached.

Thus in a first broad embodiment this invention seeks to provide a process for the preparation of a partially hydrolysed protein nutrient supplement from a raw protein containing material comprising the following steps in sequence:

(a) pretreating raw protein containing material in water to remove non-protein substances and to provide a mixture containing from about 5% to about 15% protein in water;

(b) hydrolysing the mixture from step (a) with a suitable enzyme composition at a suitable temperature;

(c) monitoring the degree of hydrolysis of the protein during the hydrolysis in step (b) until a degree of hydrolysis of from about 20% to about 35% is reached;

(d) terminating the hydrolysis reaction when the desired degree of hydrolysis has occurred by inactivating the enzyme composition;

(e) recovering a partially hydrolysed vegetable protein nutrient supplement; and

(f) if required, adjusting the pH of the resulting partially hydrolysed vegetable protein nutrient supplement to from about pH 7.0 to about pH 7.5 with an organoleptically acceptable acid or base.

Preferably the enzyme composition used in step (b) is at least one protease enzyme, and the hydrolysis temperature is from about 50° C. to about 55° C. More preferably, two protease enzymes are used.

Preferably, the hydrolysis reaction is terminated at a degree of hydrolysis of from about 25% to about 29%. More preferably, the hydrolysis reaction is terminated at a degree of hydrolysis of about 27%.

Preferably, the protein material is chosen from the group consisting of soy bean protein, wheat gluten, and corn (maize) gluten. More preferably, the protein material is soy bean protein.

Preferably, the degree of hydrolysis is monitored by osmometric readings.

Preferably, in step (a) the raw protein containing material is mixed with demineralised water.

The present invention thus relates the production of a useful and acceptable partially hydrolysed protein nutrient supplement. By using a controlled enzymatic hydrolysis, in particular a controlled protease enzymatic hydrolysis using at least one protease enzyme, it is possible to improve the properties of the protein material without adversely impacting its food or nutritional value. It is thus possible to overcome the difficulties of taste associated with soy bean based products, and to use other proteinaceous materials of vegetable origin which hitherto have not been used. The end product retains as much, and as many, proteins found, for example, in animal red blood cell material but without having to use any animal based protein material. Thus the partially hydrolysed protein supplement of this invention more or less imitates a material derived from animal protein. As will be discussed in more detail below, the partially hydrolysed protein nutrient supplement can be used together with a pharmacologically acceptable carrier as part of a dietary supplement formulation including other substances, for example isoflavone glucosides.

The following description of one embodiment of the process steps for convenience deals primarily with processing soy bean protein. The process is however to be understood not to be so limited, and can be applied to other protein materials.

In step (a) the soy bean protein material is more or less substantially freed from other materials. A convenient method is as follows. The raw material is mixed with water to provide a suspension containing from about 50 g/L to about 150 g/l protein mixture. For soy bean protein, a protein content of from about 70 g/L to about 100 g/L has been found satisfactory. The amount of water used depends to some extent on the water binding capacity of the non-proteinaceous materials present: a high water binding capacity indicates a lower amount of raw material. The raw material-water mixture is wet milled followed by jet cooking by spraying onto a hot rotating surface. Alternatively, the cooking can be effected by heating to a temperature of about 85C for a short period of time. It is preferred that the water used be demineralised. This will provide a mixture of more or less purified protein material containing about 80 g/L protein material at a temperature of from about 50° C. to about 55° C. which can be used directly for step (b).

In step (b) the isolate from step (a) is subjected to an enzymatic hydrolysis under essentially conventional conditions. For soy bean and red blood cell material a protease enzyme has been found to be satisfactory, such as Alcalase 0.6 L or Protamex (in granulated form), available from Nordisk Biochem of North America, Franklinton City, N.C., USA and from Henkel Laboratories, Canada. It is also possible to use more than one enzyme, for example two different endoprotease enzymes in sequence. The hydrolysis temperature is generally in the range for which the enzyme is active, which is usually from about 50° C. to about 55° C. The hydrolysis is carried out at the pH indicated by the enzyme suppliers, which is generally in the range of from about pH 7.0 to about pH 8.5. The slurry pH is adjusted if required with sodium hydroxide solution. The hydrolysis is carried out at a concentration of from about 70 g/l to about 80 g/L, the slurry from step (a) being diluted with water if required.

When the protein material is heated typically to a temperature of between about 50° C. and 55° C. with the protease enzyme in step (b), the degree of hydrolysis is monitored. When the hydrolysis is terminated at a degree of hydrolysis of about 27% the partially hydrolysed proteins stay in suspension in the liquid as a colloid-like solution; most of the solid waste material floats to the top as a viscous mass, and is removed by skimming.

In step (c) the progress of the enzymatic hydrolysis is monitored as a function of time. Of the several techniques that can be used for this purpose, osmolality measurements appear to be both the most effective and can be made relatively quickly.

The time span required for the hydrolysis of step (b) is subject to several variables; a typical time period is from about 12 hours to at least about 120 hours. With soy bean protein the time required can depend on both the soy bean defatting technique used, and whether the defatted protein material has been allowed to age and become somewhat dried; a shorter hydrolysis time is required for softer undried material.

In step (d) when the degree of hydrolysis has reached around 27%, the hydrolysis reaction is terminated by conventional means which will deactivate the enzyme, for example by a heat treatment step at about 85° C. for at least two minutes; generally deactivation is complete in less than about five minutes.

On termination of the hydrolysis with protease enzyme, the product is recovered in step (e). After the floating viscous materials have been removed by skimming, the remaining liquid is transferred to a clean container, typically a stainless steel pan, and allowed to cool to from about 35° C. to about 38° C.; most of the partially hydrolysed protein precipitates out of solution. After decantation of the remaining supernatant liquid, the partially hydrolysed protein material is dried under aseptic conditions, for example by heating at a temperature of about 38° C.

Alternatively, in step (e) any insoluble material, such as insoluble proteins, is removed by filtration or centrifugation. The product can also be subjected to a treatment with activated carbon, to remove any remaining undesirable fragments from the hydrolysis step. The water content of the product can also be reduced by boiling in an open pan, or by any other suitable process, such as the use of a rotary film evaporator, or by spray drying. Water removal can be assisted by the use of vacuum, for example in a rotary film evaporator.

Finally, in step (f) the pH of the product is adjusted to a physiologically acceptable level with a pharmacologically acceptable reagent, so that it is in the range of from pH 7.0 to pH 7.5. If the pH is too high, it can be adjusted using hydrochloric acid; if it is too low it can be adjusted with sodium hydroxide.

The thus obtained partially hydrolysed protein product can be used either in liquid form, or it can be-dried by spray drying to a fine powder.

The most common protein source useful in the process of this invention is soy bean protein. Other vegetable protein materials that can be used are wheat and corn (maize) protein, which can contain from about 70% to about 85% of protein, the remainder being mostly triglycerides, starch, and some fibre carbohydrates. However, wheat gluten has a significant water binding capacity, and therefore is often difficult to mix with water. To reduce this difficulty, some of the enzyme is added to the water prior to adding the wheat gluten.

The partially hydrolysed protein nutrient supplement of this invention can be used in either dry or liquid forms in food processing. It can also be used in dietary supplement preparation, for example by the addition of some specific amino acids, lactic acid, vitamin E in its natural form, some isoflavone compounds such as genistein, daidzein and glycitien, as the glucoside form, cyanocobolamine, thymosin and other nutritionally advantageous substances such as ones responsible for improved cell walls, and improved blood cell counts.

### EXAMPLE

Mixing.

5 kg of defatted soy bean protein, obtained from Central Soya Co, North Carolina, USA was mixed with demineralise water to provide a slurry having a protein content of 70 g/L(7%). Heat Treatment.

The mixture was heated to 85° C., held at this temperature for one minute, and then cooled to 50° C.

Enzymatic Hydrolysis.

The pH of the mixture was adjusted to be between pH 8.0 to pH 8.5 with 4N sodium hydroxide solution. The hydrolysis was initiated with an endoprotease, using Alcalase, available from Nordisk Biochem, Franklinton City, N.C., USA. The amount of Alcalase used was 1.5 g/L . This dosage is estimated to be about 1.5% by weight of the protein present. When the pH was observed to have decreased to about pH 7.0, a second endoprotease was added, using Neutrase, available from Novo-Nordisk, USA. The amount of Neutrase used was 0.5 g/L, which is estimated to be about 0.5% by weight of the protein present. The degree of hydrolysis of the protein was measured by osmolality measurements. After 12 hours the osmolality measurements indicated a degree of hydrolysis of 27%. Hydrolysis Termination.

The hydrolysis reaction was terminated by heating the mixture to 80° C.-85° C., holding it at this temperature for about 2 minutes, and then cooling to between 50° C. and 55° C.

Product Recovery.

After removal of floating solid waste by skimming, the mixture containing the partially hydrolysed protein was centrifuged to remove suspended material. The centrifuge supernatant was then further clarified by ultrafiltration, using a Whatman 40 (Trade Mark) ultrafiltration membrane. During ultrafiltration the temperature was monitored and maintained at about 25° C. The permeate was collected, and the retained material discarded. The partially hydrolysed protein was recovered by spray drying at an evaporation rate of 1.5L water per hour.

The partially hydrolysed protein was obtained as a microfine yellowish powder. It is soluble in demineralised water to a concentration of 2.5 mg/L.

What is claimed is:

1. A process for the preparation of a partially hydrolysed protein nutrient supplement from a vegetable raw protein containing material comprising the following steps in sequence:
(a) pretreating a raw protein containing material in water to remove non-protein substances and to provide a mixture containing from about 5% to about 15% protein in water;
(b) hydrolysing the mixture from step (a) with a suitable enzyme composition at a suitable temperature;
(c) monitoring the degree of hydrolysis of the protein during the hydrolysis in step (b) until a degree of hydrolysis of from about 20% to about 35% is reached;
(d) terminating the hydrolysis reaction when the desired degree of hydrolysis has occurred by inactivating the enzyme composition;
(e) recovering a partially hydrolysed vegetable protein nutrient supplement; and
(f) if required, adjusting the pH of the resulting partially hydrolysed vegetable protein nutrient supplement to be from about pH 7.0 to about pH 7.5 with an organoleptically acceptable acid or base.

2. A partially hydrolysed protein nutrient supplement prepared according to the process of claim 1.

3. A process according to claim 1 wherein the enzyme composition used in step (b) is at least one protease enzyme, and the hydrolysis temperature is from about 50° C. to about 55° C.

4. A process according to claim 1 wherein the enzyme composition used in step (b) is one protease enzyme, and the hydrolysis temperature is from about 50° C. to about 55° C.

5. A process according to claim 1 wherein the enzyme composition used in step (b) is two protease enzymes, and the hydrolysis temperature is from about 50° C. to about 55° C.

6. A process according to claim 1 wherein the hydrolysis reaction is terminated at a degree of hydrolysis of from about 25% to about 29%.

7. A process according to claim 6 wherein the hydrolysis reaction is terminated at a degree of hydrolysis of about 27%.

8. A process according to claim 1 wherein the protein material is chosen from the group consisting of soy bean protein, wheat gluten, and corn(maize) gluten.

9. A process according to claim 8 wherein the protein material is soy bean protein.

10. A process according to claim 1 wherein the degree of hydrolysis is monitored by osmolality readings.

11. A process according to claim 1 wherein in step (a) the raw protein containing material is mixed with demineralised water.
